(19) United States
(12) Patent Application Publication
(10) Pub. No.: US 2009/0232754 A1
(43) Pub. Date: Sep. 17, 2009

(54) ENHANCED PHOTOSTABILITY OF SUNCARE COMPOSITIONS CONTAINING AVOBENZONE

(75) Inventors: Thomas A. Meyer, Germantown, TN (US); Donathan G. Beasley, Memphis, TN (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering-Plough Healthcare Products, Inc.

(21) Appl. No.: 12/392,465

(22) Filed: Feb. 25, 2009

Related U.S. Application Data
(60) Provisional application No. 61/031,950, filed on Feb. 27, 2008.

(51) Int. Cl. A61K 8/35; A61K 31/195; A61Q 17/04; A61K 33/30

(52) U.S. Cl. 424/59; 514/567; 424/642

(57) ABSTRACT

The photostability of avobenzone in a sunscreen composition is stabilized against photodegradation using menthyl anthranilate.

Some example embodiments of the present invention generally relate to compositions for skin application to protect against harmful effects of ultraviolet radiation, particularly the effects of solar radiation. This application claims priority from U.S. provisional patent application Ser. No. 61/031,950 filed Feb. 27, 2008.

### FIELD OF THE INVENTION

### Background

Avobenzone (trade names Parsol® 1789, Eusolex® 9020, Escalol® 517 and others, INCI Butyl Methoxydibenzoylmethane) is an oil soluble ingredient used in sunscreen products to absorb the full spectrum of UV-A rays. It is a dibenzoylmethane derivative. Its ability to absorb ultraviolet light over a wider range of UVA wavelengths than many organic sunscreen agents has led to its use in many commercial preparations marketed as “broad spectrum” sunscreens.

Avobenzone has the chemical name 1-(4-methoxyphenyl)-3-(4-tert-butylphenyl)propane-1,3-dione (CAS Registry No. 70356-09-1). The molecular weight of avobenzone is 310.39 and it has the following chemical structure:

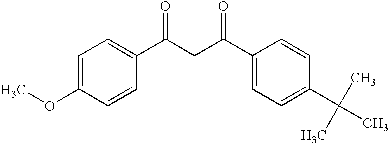

Avobenzone, as a sunscreen active, has potential to degrade chemically when exposed to ultraviolet radiation (UVR) after prolonged exposure, e.g., become photounstable. Chemical degradation robs avobenzone of its ability to absorb UVR and hence destroys its ability to protect skin against damaging UV rays when it is used as a sunscreen active in sunscreen products. Avobenzone can be maintained intact chemically as an absorber of UVR simply by adopting recognized formulation strategies to incorporate avobenzone into a sunscreen product. When formulated into a product correctly, avobenzone remains intact chemically even over prolonged exposures to UVR.

Formulation strategies to optimize avobenzone's photostability include (1) removal of incompatible ingredients, like octinoxate; (2) leveraging other sunscreen actives for their ability to enhance avobenzone's photostability; and (3) using non-sunscreen ingredients that have capacity to photostabilize avobenzone through energy transfer mechanisms, such as diethylhexyl-2,6-napthalate (known as Corapan® TQ) or diethylsyringylidene malonate (known as Oxynex® ST).

U.S. Pat. No. 7,244,416, titled “Stabilized Photoprotective Composition” to Meyer et al, generally describes a decrease in the photostability of avobenzone, particularly when it is combined with zinc oxide and that avobenzone's photostability could be enhanced by addition of phenylbenzimidazole sulfonic acid.

### SUMMARY

Applicants have found that sunscreen active menthyl anthranilate, also known as meradimate, can enhance photostability of avobenzone in sunscreen compositions exposed to UV radiation for extended periods of time. Importantly, this result is demonstrated in the absence of other agents used to photostabilize avobenzone, such as phenylbenzimidazole sulfonic acid.

One example embodiment of the invention encompasses a composition including avobenzone and menthyl anthranilate, wherein menthyl anthranilate is present in a sufficient amount to stabilize the avobenzone against photodegradation.

Another example embodiment of the invention encompasses a method for protecting the skin against ultraviolet radiation, including applying to the skin an effective amount of a composition comprising avobenzone and menthyl anthranilate, wherein menthyl anthranilate is present in a sufficient amount to stabilize the avobenzone against photodegradation.

Yet another example embodiment of the invention encompasses a method for improving the photostability of avobenzone in a composition including avobenzone and menthyl anthranilate, wherein menthyl anthranilate is present in a sufficient amount to stabilize the avobenzone against photodegradation.

### DETAILED DESCRIPTION OF EXAMPLE EMBODIMENTS

Sunscreening compositions generally are permitted to contain only the active ingredients that have been approved by governmental authorities, and frequently those authorities also specify the amounts of each approved ingredient that are permitted to be present in a product. For purposes of the present invention, a “sunscreen active agent” or “sunscreen active” shall include all of those materials, singly or in combination, that are regarded as acceptable for use as active sunscreening ingredients based on their ability to absorb and/or dissipate UV radiation. Such compounds are generally described as being UV-A, UV-B, or UV-A/UV-B active agents. Approval by a regulatory agency is generally required for inclusion of active agents in formulations intended for human use. Those active agents which have been or are currently approved for sunscreen use in the United States include organic and inorganic substances including, without limitation, para aminobenzoic acid, avobenzone, cinoxate, dioxybenzone, homosalate, octocrylene, octyl methoxycinnamate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum. Examples of additional sunscreen actives that have not yet been approved in the US but are allowed in formulations sold outside of the US include ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. However, as the list of approved sunscreens is currently expanding, those of ordinary skill will recognize that the invention is not limited to sunscreen active agents currently approved for human use but is readily applicable to those that may be allowed in the future.

Section 352.20 of the same Title 21 describes the permitted combinations of ingredients; in general, each active ingredient in a permitted combination is required to be present in at least a sufficient amount to contribute an SPF value of 2, so an amount making this contribution is considered herein to be the minimum “sunscreening-effective” concentration of an active ingredient. The regulations prescribe maximum concentrations of 3 percent avobenzone, and 25 percent zinc oxide. Some countries allow the use of other active ingredients noted above and these are also suitable for inclusion as components of the compositions of this invention. In addition, the permitted concentrations of active ingredients vary somewhat by country.

The official adopted name in the United States for octyl methoxycinnamate is “octinoxate,” and the official name for octyl salicylate is “octisalate”. Z-COTE HP1® is a particle of micro-fine zinc oxide, coated with dimethicone. Alternatively, ZinClear-IM™ may be used. ZinClear-IM™ is a type of zinc oxide that has an average particle size >1.0 micron but yet is highly transparent. ZinClear-IM™ is hydrophobically modified and is available in the form of a dispersion using common cosmetic emollients, such as C₁₂-C₁₅ alkyl benzoate or caprylic/capric triglycerides. KELTROL® CG F is a xanthan gum biopolymer. DOW CORNING 200® Fluid, is a polydimethylsiloxane. It is sold in wide range of viscosity, i.e. 10 cSt to 60,000 cSt. Preferably, having a viscosity of 350 cSt. VEEGUM ULTRA® is magnesium aluminum silicate and purified smectite clay.

Title 21, in Section 352.3, defines the term “Sun Protection Factor,” typically abbreviated as “SPF,” which is determined by testing unprotected and sunscreen-protected skin using standardized intensities and amounts of ultraviolet radiation. Protected skin for this testing has been treated by an application of a sunscreen product at the rate of 2 mg/cm², and it is intended that the compositions of this invention will be applied by a user at that same rate to achieve the rated protection levels.

### Sunscreen Composition

One example embodiment of the present invention encompasses a composition comprising avobenzone and menthyl anthranilate. Menthyl anthranilate has the structure:

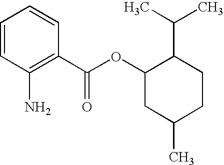

(C₁₇H₂₅NO₂ 275.39) and is also referred to as menthyl-O-aminobenzoate and anthranilic acid,p-menth-3-yl ester.

The inventors have surprisingly discovered that when menthyl anthranilate is present in a sufficient amount, avobenzone's photostability is increased even over long exposures to ultraviolet radiation. The increased photostability results are unexpected in view of the fact that menthyl anthranilate's chemical structure, a derivative of amino benzoate, is similar to sunscreen actives that are known to destabilize avobenzone, such as PABA and Padimate O, which are para-amino benzonates. In comparison, menthyl anthranilate is an ortho-amino benzoate.

The composition can also contain an emulsifier, typically hydrogenated palm glycerides or C₈-C₃₄ fatty alcohols. Preferably, the C₈-C₃₄ fatty alcohol is capry alcohol, capic alcohol, myristyl alcohol, cetyl alcohol, stearyl alcohol, arachidyl alcohol, or cetearyl alcohol. The emulsifier can also be a phosphate-based emulsifiers such as potassium octyl phosphate, potassium nonyl phosphate, potassium decyl phosphate, potassium undecyl phosphate, potassium lauryl phosphate, potassium myristyl phosphate, potassium cetyl phosphate, potassium stearyl phosphate, dicetyl phosphate, ceteth-10 phosphate, ceteth-20 phosphate, or ceteth-30 phosphate. More preferably, the phosphate-based emulsifier is a mixture of potassium cetyl phosphate and hydrogenated palm glycerides or a mixture of cetearyl alcohol, dicetyl phosphate and ceteth-10 phosphate. Typically, the emulsifier, in particular a phosphate-based emulsifier, is present from about 1 to about 6 weight percent of the composition. Preferably, it is present as about 5 weight percent of the composition.

These compositions may further contain at least one additional sunscreen active besides avobenzone and menthyl anthranilate. Representative sunscreen actives are recited above. Typically, the sunscreen actives are octisalate, homosalate, octocrylene, oxybenzone or combinations thereof.

Typically, the concentration of avobenzone is from about 1 to about 3 weight percent of the composition. Preferably, the concentration of avobenzone is from about 2 weight percent of the composition. Typically, the concentration of zinc oxide is from about 5 to about 25 weight percent of the composition. Preferably, the concentration of zinc oxide is about 5 to about 15 weight percent of the composition. More preferably, the concentration of zinc oxide is about 5 to about 10 weight percent of the concentration. Most preferably, the concentration of zinc oxide is about 5 weight percent of the composition.

Useful sunscreen compositions according to the present invention can be prepared in the form of fluid suspensions, gels, sticks and others, utilizing formulation parameters known in the art. However, the compositions of the invention are more typically emulsions, such as lotions and creams. In many instances it will be preferred to prepare emulsions of the oil-in-water type, since these can appear to the skin as being aqueous in character and therefore give a more pleasant sensation while they are being applied. However, the water-in-oil type of emulsion is also useful since, after application, contained water evaporates; both types of emulsions will leave a nonaqueous residue on the skin.

Emulsion compositions of the invention generally contain, in addition to the active sunscreening agents, water and at least one emulsifier. One or more other types of components will frequently also be present, such as, without limitation, emulsion builders, emollients, humectants, dry-feel modifiers, waterproofing agents, antimicrobial preservatives, antioxidants, chelating agents, fragrances, colorants and insect repellents.

### Emulsions/Emulsifiers

A stable emulsion is a mixture of at least two immiscible liquids, e.g., liquids that are not mutually soluble, but in the presence of an emulsifier, are mechanically agitated and shaken so thoroughly together that one liquid forms drops in the other one, giving the mixture the appearance of a homogeneous liquid. These liquids may include materials which are solid or solid-like at room temperature, but will liquefy at a higher temperature during processing. The presence of an emulsifier enables one of the immiscible liquids to remain in a continuous form, while allowing the other immiscible liquid to remain in a dispersed droplet form. Thus, one function of an emulsifier, a stabilizing compound, is to assist in the production of a stable emulsion. A secondary function of emulsifiers is to provide a thickening or “bodying” to an emulsion. Typically, emulsifiers are molecules with non-polar and polar parts that are able to reside at the interface of the two immiscible liquids. As used herein in reference to the water-in-oil emulsifiers, the term “HLB value” means the hydrophilic/lipophilic balance. The HLB value has been used by those skilled in the emulsion art for selecting emulsifiers useful to prepare, inter alia, water-in-oil emulsions. See U.S. Pat. No. 4,177,259 and references cited therein.

An oil-in-water (o/w) emulsion is a mixture where “oil,” or water-insoluble liquid, droplets (the discontinuous phase) are dispersed in a continuous aqueous phase. A water-in-oil (w/o) emulsion is a mixture where aqueous phase droplets (the discontinuous phase) are dispersed in “oil” (a continuous water-insoluble phase). Preferably, the example composition is an oil-in-water emulsion where the oil-soluble actives are combined to form the oil phase, prior to mixture with the water phase. The type of emulsion formed, oil-in-water (o/w) or water-in-oil (w/o), is sometimes determined by the volume ratio of the two liquids provided the ratio is sufficiently high. For example, with 5% water and 95% oil (an o/w phase ratio of 19), the emulsion likely will become w/o. For moderate phase ratios (generally <3), the type of emulsion is decided by several factors, such as order of addition or type of emulsifier. One liquid slowly added to a second liquid with agitation usually results in the second liquid being the continuous phase. Another factor is preferred solubility of the emulsifier, as the phase in which the emulsifier is more soluble will likely be continuous.

More complex emulsions such as double emulsions are formed where an emulsion is dispersed in a continuous phase. For example, in an oil-in-water-in-oil (o/w/o) emulsion, the water in a continuous water phase containing dispersed oil droplets, is itself dispersed in a continuous oil phase. Similarly, in a water-in oil-in water (w/o/w) emulsion, the oil in a continuous phase containing dispersed water droplets, is itself dispersed in a continuous water phase. These more complex emulsions find use as a system for slow delivery, extraction, etc.

Typical suitable emulsifiers having an HLB value about 1 to about 7 include sorbitan monooleate, sorbitan sesquioleate, sorbitan isostearate, sorbitan trioleate, PEG-22/dodecyl glycol copolymer, PEG-45/dodecyl glycol copolymer, polyglyceryl-3-diisostearate, polyglycerol esters of oleic/isostearic acid, polyglyceryl-6 hexaricinolate, polyglyceryl-4 oleate, polyglyceryl-4 oleate/PEG-8 propylene glycol cocoate, oleamide DEA, sodium glyceryl oleate phosphate and hydrogenated vegetable glycerides phosphate.

During preparation of the emulsion, an acid or a base may be added to adjust the pH of one or more ingredients, e.g., to adjust the viscosity of a polymeric thickener, prior to its inclusion in the sunscreen composition. For example, triethanolamine, a base, can be used to increase the pH of the water phase and consequently, modify the desired viscosity of the emulsion. The sunscreen can have a pH of about 6.5 to about 8, preferably from about 6.5 to about 7.5, more preferably the pH of the sunscreen is neutral, i.e., about 7.0. When present together in a composition, certain ingredients such as triethanolamine and stearic acid can form an emulsifier. As is well known, inorganic salts such as sodium chloride also are frequently included in emulsion compositions to obtain desired product stability and other physical properties.

Conveniently, one or more emulsifiers can be used in the example compositions in amounts ranging from about 0.05 to about 20 weight percent of the emulsion, preferably from about 0.1 to about 15%, more preferably from about 5 to about 10%.

### Water

Water is employed in amounts effective to form the emulsion. For hydrophilic or water-loving ingredients, the amount of water should be sufficient to at least solubilize these ingredients. For hydrophobic or water-repelling ingredients, the water should be employed in amounts to serve as the continuous phase of an oil-in water emulsion. Thus, amount of water in the emulsion or composition can range from about 2 to 95 weight %, preferably from 50 to 85%. It frequently is desirable to use purified water, to enhance the predictability of product characteristics.

### Emollients

An emollient is an oleaginous or oily substance which helps to smooth and soften the skin, and may also reduce its roughness, cracking or irritation. Typical suitable emollients include mineral, oil, having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural sonora jojoba oils, safflower oil, corn oil, liquid lanolin, cottonseed oil and peanut oil.

Other suitable emollients include squalane, castor oil, polybutene, odorless mineral spirits, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, octyl palmitate which is commercially available as Lexol EHP, tradename of Inolex Co. of Philadelphia, Pa. U.S.A., isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glycerol, ricinoleates of alcohols and polyalcohols such as those of isopropyl adipate, hexyl laurate and octyl dodecanoate.

Other suitable emollients which are solids or semi-solids at ambient temperatures may be used in amounts sufficient to provide liquid topical compositions. Such solid or semi-solid cosmetic emollients include hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. One or more emollients can optionally be included in the example sunscreen emulsion in an amount ranging from about 10 to about 50 weight %, preferably about 20 to about 40%.

### Humectants

A humectant is a moistening agent that promotes retention of water due to its hygroscopic properties. Suitable humectants include urea, glycerin, polymeric glycols such as poyethylene glycol and polypropylene glycol, and sorbitols. One or more humectants can optionally be included in the in the example sunscreen in amounts from about 1 to 10 weight %.

### Dry-Feel Modifiers

A dry-feel modifier is an agent which, when incorporated in an emulsion, imparts a “dry feel” to the skin when the emulsion dries. Dry-feel modifiers may also reduce sunscreen migration on the skin. Dry feel modifiers can include starches, talc, kaolin, chalk, zinc oxide, silicone fluids, inorganic salts such as barium sulfate and sodium chloride, C₆ to C₁₂ alcohols such as octanol; sulfonated oils; surface treated silica, precipitated silica, fumed silica such as Aerosil® available from the Degussa Inc. of New York, N.Y. U.S.A. or mixtures thereof; dimethicone, a mixture of mixture of methylated linear siloxane polymers, available as DC200 fluid, tradename of Dow Corning, Midland, Mich. U.S.A. One or more dry-feel modifiers can optionally be included in the sunscreen in amounts ranging from 0.01 to about 20 weight %, more preferably from about 0.5 to about 6 weight %.

### Waterproofing Agents

A waterproofing agent is a hydrophobic material that imparts film forming and waterproofing characteristics to an emulsion. Typical suitable waterproofing agents include copolymers derived from polymerization of octadecene-1 and maleic anhydride in accordance with the published procedures such as those in U.S. Pat. No. 3,860,700 and Reissue No. 28,475. A preferred waterproofing agent is a polyanhydride resin, also known as PA-18, tradename of the Chevron Chemicals Co., San Francisco, Calif. U.S.A. Another preferred waterproofing agent is a copolymer of vinyl pyrollidone and eicosene monomers such as Ganex Polymer, tradename of ISP Inc. of Wayne, N.J. U.S.A.

By the term “waterproofing effective amount of at least one waterproofing agent” means the waterproofing agent(s) is used in amounts effective to allow the sunscreen to remain on the skin after exposure to circulating water for at least 80 minutes using the procedures described in “Sunscreen Drug Products for OTC Human Use”, Federal Register, Vol. 43, Aug. 25, 1978, Part 2, pp 38206-38269. One or more waterproofing agents can optionally be included in the sunscreen composition in an amount ranging from about 0.01 to about 10.0 weight percent, preferably about 1.0 to about 10.0 percent.

Examples of suitable waterproofing agents may be found in U.S. Published Patent Application No. 2005-0276833, published Dec. 15, 2005, titled “Skin care compositions” to Kevin C. Fowler.

### Antimicrobial Preservatives

An antimicrobial preservative is a substance or preparation which destroys, prevents or inhibits the multiplication/growth of microorganisms in the sunscreen composition and may offer protection from oxidation. Preservatives are used to make self-sterilizing, aqueous based products such as emulsions. This is done to prevent the development of microorganisms that may be in the product during manufacturing and distribution, and during use by consumers who may inadvertently contaminate the products. Typical preservatives include the lower alkyl esters of para-hydroxybenzoates (parabens) especially, methylparaben, propylparaben, isobutylparaben and mixtures thereof, benzyl alcohol and benzoic acid. One or more antimicrobial preservatives can optionally be included in the sunscreen composition in an amount ranging from about 0.001 to about 10 weight percent, more preferably about 0.05 to about 2 percent.

### Antioxidants

An antioxidant is a natural or synthetic substance added to the sunscreen to protect from or delay its deterioration due to the action of oxygen from the air, or to protect the skin against damage from free radicals that form due to the action of ultraviolet radiation. Typical suitable antioxidants include propyl, octyl and dodecyl esters of gallic acid, butylated hydroxyanisole (BHA) which is usually as a mixture of ortho and meta isomers, butylated hydroxytoluene (BHT), nordihydroguaiaretic acid, vitamin E, vitamin E acetate, vitamin C and alkylated parabens such as methylparaben and propylparaben. One or more antioxidants can optionally be included in the sunscreen composition in an amount ranging from about 0.001 to about 5 weight percent, preferably about 0.05 to about 2 percent.

### Chelating Agents

Chelating agents are substances used to complex or bind metallic ions in a frequently heterocylic ring structure so that the ion is held by chemical bonds from members of the ring. Suitable chelating agents include ethylene diaminetetraacetic acid (EDTA), EDTA disodium, calcium disodium edetate, EDTA trisodium, EDTA tetrasodium and EDTA dipotassium. One or more chelating agents can optionally be included in the sunscreen in amounts ranging from about 0.001 to about 0.1 weight percent.

### Fragrances

Fragrances are aromatic compounds which can impart an aesthetically pleasing aroma to the sunscreen composition. Typical fragrances include aromatic materials extracted from botanical sources (i.e. rose petals, gardenia blossoms, jasmine flowers, aloe barbadensis leaf extract (aloe vera) etc.) which can be used alone or in any combination to create essential oils. Alternatively, alcoholic extracts may be prepared for compounding fragrances. One or more fragrances can optionally be included in the sunscreen composition in an amount ranging from about 0.001 to about 10 weight percent, preferably about 0.05 to about 5 percent.

### Insect Repellents

It frequently is desirable to provide protection against biting and stinging insects, since sunscreens are used in outdoor environments. Useful insect repelling ingredients include synthetic agents such as N,N-diethyl-m-toluamide, also commonly known as “DEET,” and natural plant extracts such as citronella, geraniol and others.

### Dispensers

The sunscreen emulsions of the present invention can be stored or dispensed in any container suitable for convenient delivery, for example pouring or spraying. Such containers can include, but are not limited to, jars and bottles which permit pouring of the contents, bottles having lotion pumps, pump spray bottles and pressurized aerosol and non-aerosol canisters.

Having described the invention with reference to certain preferred embodiments, other embodiments will become apparent to one skilled in the art from consideration of the specification. It will be apparent to those skilled in the art that many modifications, both to materials and methods, may be practiced without departing from the scope of the invention. Specifically, it will be apparent to those skilled in the art that many other phosphate-based emulsifiers exist for use in development of topical skincare products and that some of these may also confer enhanced photostability to avobenzone when it is combined with zinc oxide. In addition, it will be appreciated that similar techniques and compositions may be used not only for sunscreen products, such as, suntan lotions and sprays of various types, but also for cosmetics, insect repellants and other products where sunscreen compositions may be included.

In the specification, the present invention has been described with reference to specific example embodiments thereof. The specification and drawings are accordingly to be regarded in an illustrative rather than restrictive sense. The broader spirit and scope of the invention is set out in the claims that follow the specification.

### Methods of Use

Topical application of the compositions described herein to the hair or skin of a human will provide enhanced protection against deleterious effects of ultraviolet radiation (UVR). The topical application may be by a variety of approaches, including creams, lotions, sprays, or liquids. Thus, further example embodiments of the present invention include methods for protecting human skin and/or hair against the deleterious effects of solar radiation, more particularly UVR by topically applying thereto an effective amount of the sunscreen compositions as described herein. An esthetically beneficial result of exposure of skin to UVR (i.e., light radiation wavelengths of from 280 nm to 400 nm) is the promotion of tanning of the human epidermis. Another benefit of sun exposure comes from production of vitamin D within the skin. UVR is typically divided into UV-A (light wavelengths from 320 to 400 nm) and UV-B (wavelengths ranging from 280 to 320 nm) regions. Overexposure to UV-B irradiation is generally understood to lead to skin burns and erythema. In addition, overexposure to UV-A radiation may cause a loss of elasticity of the skin and the appearance of wrinkles, promoting premature skin aging. Such irradiation promotes triggering of the erythemal reaction or amplifies this reaction in certain individuals and may even be the source of phototoxic or photoallergic reactions. It is increasingly believed that overexposure to UV-A may also lead to melanoma. Thus, the application of the compositions described herein to the skin and/or hair of an individual will provide enhanced UVR photoprotection (UV-A and/or UV-B) of the skin and/or hair of the individual.

Certain embodiments of the compositions of the invention are intended to provide a sun protection factor (SPF) rating of at least 2, with additional preferable embodiments having a sun protection factor of at least 5, at least 10, at least 15, at least 20, at least 25, at least 30, at least 35, at least 40, at least 45, at least 50, at least 55, at least 60, at least 65, at least 70, at least 75, at least 80, and at least 85. Certain embodiments of the compositions of the invention are also intended to provide the highest UV-A ratings as recently proposed by the US FDA, other wise referred to as the “four star” rating system.

Application of the compositions described herein may be at different intervals, e.g., hourly, every four hours, or every eight hours, depending on the particular composition and the level and type of activity by the wearer, with more frequent application recommended for better sun protection.

### Experimental

To investigate its stabilizing effect of menthyl anthranilate on the photostability of avobenzone, menthyl anthranilate (meradimate) was incorporated into a fixed sunscreen composition at different levels. Thin film strips of each composition were then irradiated with solar simulated UVR for up to 4 hours as described in detail below. The compositions were then analyzed for the presence of avobenzone after a given period of time.

Formula compositions were prepared as follows:

TABLE 1
Formula Compositions

| Ingredient | INCI Name | % (w/w) |
|---|---|---|
| Part A | | |
| Avobenzone | Avobenzone | 3.00 |
| Oxybenzone | Oxybenzone | 6.00 |
| Octisalate | Octisalate | 5.00 |
| Homosalate | Homosalate | 15.00 |
| Menthyl Anthranilate | Meradimate | 0 |
| | | 1.00 |
| | | 3.00 |
| | | 5.00 |
| Part B | | |
| Dermacryl 79 | Acrylates/octylacrylamide | 3.00 |
| | copolymer | |
| Part C | | |
| Glycerin | Glycerin | 1.00 |
| Aloe Vera | aloe barbadensis leaf extract | 0.01 |
| Extract | | |
| Fragrance | Fragrance | 0.20 |
| Alcohol SD-40-2 | SD alcohol 40 | q.s. to 100% |
| Anhydrous | | |

Compositions were prepared by adding ingredients of Part A (oil phase) into a container large enough to hold the entire batch and then mixed and heated to 51-57° C. to dissolve oxybenzone and avobenzone. After the oil phase was homogenous, the heat was removed but mixing continued. While mixing, Part B (Dermacryl 79) was added by slowly sprinkling it into the oil phase until it was dispersed. The alcohol from Part C was then added, followed the remainder of the ingredients of Part C in order and mixing continued until all ingredients dissolved. The compositions were then allowed to cool to room temperature.

Assessment of avobenzone's photostability was performed on four different compositions that differed only in the amount of menthyl anthranilate that were used in the formulation. The results of the photostability testing are described in Table 2 below.

TABLE 2
Photostability of Avobenzone with Meradimate

| | | Hours of UV | % Avobenzone |
|---|---|---|---|
| Formula | % Meradimate | Exposure | remaining |
| 1 | 0.00 | 0 | 96.5 |
| | | 2 | 86.6 |
| | | 4 | 65.8 |
| 2 | 1.00 | 0 | 99.5 |
| | | 2 | 92.1 |
| | | 4 | 83.2 |
| 3 | 3.00 | 0 | 100.3 |
| | | 2 | 96.7 |
| | | 4 | 89.0 |
| 4 | 5.00 | 0 | 99.5 |
| | | 2 | 93.7 |
| | | 4 | 91.4 |

The photostability of avobenzone in each emulsion was assessed after exposure to 80 joules of UV radiation (which corresponds to about 4 hours of outside sun exposure at noon in Memphis, Tenn. in June) using the method described below. The results shown in Table 2 clearly supports that addition of menthyl anthranilate at all levels to the composition containing sunscreen actives octisalate, homosalate, and avobenzone, improves photostability of avobenzone to ultraviolet radiation (UVR) up to 4 hours. Even 1% menthyl anthranilate improved avobenzone's photostability by approximately 6% after 2 hours and by 17% after 4 hours of exposure. At the 5% level, menthyl anthranilate improved avobenzone's photostability by 25%.

### Method To Assess Photostability:

Avobenzone's photostability was assessed by spreading known weights (14-18 mg) of emulsions onto glass microscope slides at an application density of about 2 mg/cm². After application, the glass slides were allowed to air dry in the dark for at least 20 minutes prior to irradiation with ultraviolet radiation (UVR). Non-irradiated emulsion-treated glass slides served as controls and were stored in the dark until extraction. Each emulsion was irradiated in triplicate at an UVR dose of 80 joules/cm². The glass slides were positioned on a turntable and irradiated at a distance of 120 cm from the source using a 1000 W Xe arc solar simulator (Spectral Energy, Washingtonville, N.Y.) filtered with a WG320 filter (Solar Light Company, Inc., Glenside, Pa.). UVR flux was measured using an OL 754 Spectroradiometer (Optronics laboratories, Inc., Orlando, Fla.) with an 8 inch integrating sphere. The UVR flux from the solar simulator was adjusted prior to each irradiation to give 20 joules of UVR (290-400 nm) over a 60 minute period plus or minus 8 minutes. The dose of 80 joules/cm² (˜14.4 MED) corresponds to exposure of about fours of midday June sun in Memphis, Tenn.

Following irradiations, both irradiated and non-irradiated product treated glass slides were placed in 4 ounce glass jars with 50 ml of isopropanol and the slides were gently rubbed with a gloved finger to ensure complete extraction of the sunscreens. Following extraction, UV absorbance of the isopropanol extracts was recorded from 290 to 400 nm using a Lambda 40 spectrophotometer (Perkin-Elmer, Wellesley, Mass.). The loss in avobenzone was monitored by following the loss in absorbance at 360 nm, which was corrected for background contributions from other sunscreen actives. Corrected absorbances at 360 nm due only to avobenzone were then used to obtain a calculated weight of lotion from a standard curve prepared for each product over a weight range of ca. 1.5-22 mg. Calculated weights were used to compute the percent avobenzone remaining after irradiation according to the following equation: % avobenzone remaining=[calculated emulsion weight/actual emulsion weight]×100%.

What is claimed is:

1. A composition comprising avobenzone and menthyl anthranilate in an amount to stabilize avobenzone against photodegradation.

2. The composition of claim 1, wherein menthyl anthranilate is present from about 1 to about 6 weight percent of the composition.

3. The composition of claim 2, wherein the composition is substantially free of additional non-sunscreen agents that photostabilize avobenzone.

4. The composition of claim 1, wherein menthyl anthranilate is present in an amount of at least 1 weight percent of the composition.

5. The composition of claim 1, wherein menthyl anthranilate is present in an amount of at least 3 weight percent of the composition.

6. The composition of claim 1, wherein menthyl anthranilate is present in an amount of at least 5 weight percent of the composition.

7. The composition of claim 1, wherein the composition further comprises at least one additional sunscreen active besides avobenzone and menthyl anthranilate.

8. The composition of claim 7, wherein the additional sunscreen active is selected from the group consisting of octisalate, homosalate, octocrylene, oxybenzone, and combinations thereof.

9. The composition of claim 1, wherein the concentration of avobenzone is from about 1 to about 3 weight percent of the composition.

10. The composition of claim 9, wherein the concentration of avobenzone is about 2 weight percent of the composition.

11. The composition of claim 1, further comprising zinc oxide.

12. The composition of claim 11, wherein the concentration of zinc oxide is from about 5 to about 25 weight percent of the composition.

13. The composition of claim 11, wherein the concentration of zinc oxide is from about 5 to about 15 weight percent of the composition.

14. The composition of claim 11, wherein the concentration of zinc oxide is from about 5 to about 10 weight percent of the composition.

15. The composition of claim 11, wherein the concentration of zinc oxide is about 5 weight percent of the composition.

16. The composition of claim 1, wherein the composition is in the form of an emulsion.

17. The composition of claim 16, wherein the emulsion is an oil-in-water emulsion.

18. The composition of claim 16, wherein the emulsion is a water-in-oil emulsion.

19. A method for protecting the skin against ultraviolet radiation, comprising applying to the skin an effective amount of a composition comprising avobenzone and menthyl anthranilate wherein the menthyl anthranilate is present in a sufficient amount to stabilize the avobenzone against photodegradation.

20. The method of claim 19, wherein the concentration of avobenzone in the composition is from about 1 to about 3 weight percent of the composition.

21. The method of claim 19, wherein menthyl anthranilate is present from about 1 to about 6 weight percent of the composition.

22. The method of claim 19, wherein the composition is substantially free of additional non-sunscreen agents that photostabilize avobenzone.

23. The method of claim 19, wherein menthyl anthranilate is present in an amount of at least 1 weight percent of the composition.

24. The method of claim 19, wherein menthyl anthranilate is present in an amount of at least 3 weight percent of the composition.

25. The method of claim 19, wherein menthyl anthranilate is present in an amount of at least 5 weight percent of the composition.

26. The method of claim 19, wherein avobenzone is stabilized against photodegradation for a period of at least 2 hours.

27. The method of claim 19, wherein avobenzone is stabilized against photodegradation for a period of at least 4 hours.

28. A method for improving the photostability of avobenzone in a composition including avobenzone, the method comprising adding menthyl anthranilate to the composition in a sufficient amount to stabilize the avobenzone against photodegradation.

29. The method of claim 28, wherein the concentration of avobenzone is from about 1 to about 3 weight percent of the composition.

30. The method of claim 28, wherein avobenzone is stabilized against photodegradation for a period of at least 2 hours.

31. The method of claim 28, wherein avobenzone is stabilized against photodegradation for a period of at least 4 hours.

32. The method of claim 28, wherein the composition is substantially free of additional non-sunscreen agents that photostabilize avobenzone.
